(19) United States
(12) Patent Application Publication
(10) Pub. No.: US 2018/0250285 A1
(43) Pub. Date: Sep. 6, 2018

(54) COMBINATION OF LAQUINIMOD AND PRIDOPIDINE FOR TREATING NEURODEGENERATIVE DISORDERS, IN PARTICULAR HUNTINGTON'S DISEASE

(71) Applicant: Michael HAYDEN, Petach-Tikva (IL)

(72) Inventor: Michael HAYDEN, Petach-Tikva (IL)

(73) Assignee: Teva Pharmaceutical Industries Ltd., Petach-Tikva (IL)

(21) Appl. No.: 15/974,112

(22) Filed: May 8, 2018

Related U.S. Application Data
(63) Continuation of application No. 15/801,703, filed on Nov. 2, 2017.
Application No. 15/801,703 is a continuation of application No. 15/655,580, filed on Jul. 20, 2017.
Application No. 15/655,580 is a continuation of application No. 14/426,327, filed on Mar. 5, 2015, now abandoned.
(60) Provisional application No. 61/879,004, filed on Sep. 17, 2013.
(60) Provisional application No. 61/706,695, filed on Sep. 27, 2012.

(51) Int. Cl. A61K 31/4704; B65D 81/32; A61K 31/451

(52) U.S. Cl. CPC A61K 31/4704; B65D 81/32; A61K 31/451; A61K 2300/00

(57) ABSTRACT

This invention provides a method of treating a patient afflicted with a neurodegenerative disorder, e.g., Huntington's disease (HD), comprising administering to the patient laquinimod as an add-on therapy to or in combination with pridopidine. This invention also provides a package and a pharmaceutical composition comprising laquinimod and pridopidine for treating a patient afflicted with a neurodegenerative disorder, e.g., HD. This invention also provides laquinimod for use as an add-on therapy or in combination with pridopidine in treating a patient afflicted with a neurodegenerative disorder, e.g., HD. This invention further provides use of laquinimod and pridopidine in the preparation of a combination for treating a patient afflicted with a neurodegenerative disorder, e.g., HD.

This application claims priority of U.S. Provisional Application No. 61/879,004, filed Sep. 17, 2013, and U.S. Provisional Application No. 61/706,695, filed Sep. 27, 2012, the entire contents of which are hereby incorporated by reference herein.

Throughout this application, various publications are referred to by first author and year of publication. Full citations for these publications are presented in a References section immediately before the claims. Disclosures of the documents and publications cited and those in the References section are hereby incorporated by reference in their entireties into this application in order to more fully describe the state of the art as of the date of the invention described herein.

### BACKGROUND

Huntington's disease (HD) is an inherited disease of the central nervous system (CNS) that is characterized by chorea and progressive cognitive deterioration. Symptoms and signs of HD develop insidiously, starting at about age 35-50 but can develop before adulthood. Dementia or psychiatric disturbances (e.g., depression, apathy, irritability, anhedonia, antisocial behavior, full-blown bipolar or schizophreniform disorder) can develop before or simultaneously with the movement disorder. Symptoms of HD also include abnormal movements, such as myoclonic jerks or irregular movements of extremities, a lilting gait, facial grimacing, ataxia and inability to sustain motor act (motor impersistence) such as tongue protrusion. As the disease progresses, walking and swallowing become more difficult and dementia becomes more severe. Most HD patients will eventually require institutionalization, and death usually occurs 13-15 years after the symptoms begin, usually due to an intercurrent infection (Tyagi et al., 2010; The Merck Manual).

HD is an autosomal dominant disorder resulting from a gene mutation causing abnormal repetition of the DNA sequence CAG which codes for the amino acid glutamine. The resulting huntingtin protein (Htt) is a mutant huntingtin (mHtt) with an expanded stretch of polyglutamine residues, leading to the disease via unknown mechanisms (The Merck Manual).

There is currently no cure for HD. In addition, tetrabenzaine is the only medication currently approved by the Food and Drug Administration (FDA) to treat the symptoms of Huntington's disease. However other supportive therapies are currently available to manage the symptoms. Symptomatic treatment of Huntington's disease involves use of dopamine antagonists, presynaptic dopamine depleters, antidepressants, tranquillizers, anxiolytic benzodiazepines, anticonvulsants and antibiotics. Chorea and agitation may be partially suppressed by antipsychotics (e.g., chloropromazine 25-300 mg po id, haloperidol 5-45 mg po bid); dose is increased until intolerable or undesirable adverse effects (e.g., lethargy, parkinsonism) occur. Alternatively, tetrabenazine may be used. The dose starts at 12.5 mg po once/day, and is subsequently increased (to 12.5 mg bid in the second week, 12.5 tid in the third week, up to a total of 100 mg/day divided into 3 doses) until intolerable adverse effects (e.g., sedation, akathisias, parkinsonism, depression) occur or chorea resolves (Tyagi et al., 2010; The Merck Manual).

Several medications including baclofen, idebenone and vitamin E have been studied in clinical trials with limited samples. Some experimental therapies for HD have aimed to reduce glutamatergic neurotransmission via the N-methyl-D-aspartate receptor and to bolster mitochondrial energy production. However, currently no other drug has been recommended for HD (Tyagi et al., 2010; The Merck Manual).

Pridopidine (4-(3-(Methylsulfonyl)phenyl)-1-propylpiperidine)

Pridopidine (ACR16, Huntexil®, 4-[3-(methylsulfonyl)phenyl]-1-propyl-piperidine]) is a dopamine receptor mixed antagonist/agonist (U.S. Patent Application Publication No. 2011/0206782). Pridopidine shows benefits in treating neurodegenerative disorders including Huntington's Disease (Miller & Bezprozvanny 2010).

Pridopidine acts on central dopamine D2 receptors to potentially improve voluntary motor function in Huntington's disease patients (Venuto, 2012). The method of action is still not precisely known but pridopidine may stimulate or inhibit dopamine to normalize hypo- and hyper-dopaminergic behavior (Miller & Bezprozvanny 2010).

Huntexil® is the brand name for pridopidine developed by Neurosearch, Denmark to treat movement and psychiatric disorders (Miller & Bezprozvanny 2010). A recent MermaiHD Phase III clinical trial in Europe showed benefits from a treatment of 45 mg daily, or 90 mg daily dose (45 mg administered twice daily) for 6 months in Huntington's disease patients. Amounts of pridopidine up to 90 mg per day were well tolerated in Huntington's disease patients. The primary endpoint was the effect of Huntexil® on a specific subset of motor symptoms defined in the mMS at 26 weeks and was not met. However, the tertiary endpoint, UHDRS-TMS measuring changes in motor function, and individual items within the mMS (including gait and dysarthria) found a statistically significant effect of treatment (de Yebenes, 2011). Huntexil® slowed Huntington's disease symptoms and may have slowed Huntington's disease progression (Miller & Bezprozvanny 2010). The HART trial, initial Phase Ill) studies in the United States and Canada, showed a significant effect on total motor function after twice-daily doses of 45 mg over 12 weeks (NeuroSearch-The HART Study). Clinical trials in the United States are ongoing to assess the long-term safety and treatment effects (Clinical Trials: OPEN-HART, 2011).

### Laquinimod

Laquinimod is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851.

The mechanism of action of laquinimod is not fully understood. Animal studies show it causes a Th1 (T helper 1 cell, produces pro-inflammatory cytokines) to Th2 (T helper 2 cell, produces anti-inflammatory cytokines) shift with an anti-inflammatory profile (Yang, 2004; Bruck, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runstrom, 2006; Bruck, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

### Add-On/Combination Therapy

The effects of add-on or combination therapy using laquinimod and pridopidine on patients afflicted with a neurodegenerative disorder, e.g., HD, have not been reported.

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1 a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of an add-on or combination therapy of two drugs, in particular laquinimod and pridopidine, cannot be predicted until the results of a formal combination study are available.

### SUMMARY OF THE INVENTION

This invention provides a method of treating a human patient afflicted with a neurodegenerative disorder comprising periodically administering to the patient an amount of laquinimod and an amount of pridopidine, wherein the amounts when taken together are effective to treat the human patient.

This invention also provides a package comprising (a) a first pharmaceutical composition comprising an amount of laquinimod and a pharmaceutically acceptable carrier; (b) a second pharmaceutical composition comprising an amount of pridopidine and a pharmaceutically acceptable carrier; and (c) instructions for use of the first and second pharmaceutical compositions together to treat a human patient afflicted with a neurodegenerative disease.

This invention also provides laquinimod for use as an add-on therapy or in combination with pridopidine in treating a human patient afflicted with a neurodegenerative disorder.

This invention also provides a pharmaceutical composition comprising an amount of laquinimod and an amount of pridopidine for use in treating a human patient afflicted with a neurodegenerative disorder, wherein the laquinimod and the pridopidine are to be administered simultaneously or contemporaneously.

This invention also provides use of an amount of laquinimod and an amount of pridopidine in the preparation of a combination for treating a human patient afflicted with a neurodegenerative disorder wherein the laquinimod or pharmaceutically acceptable salt thereof and the pridopidine are administered simultaneously or contemporaneously.

This invention also provides a pharmaceutical composition comprising an amount of laquinimod for use in treating a subject afflicted with a neurodegenerative disorder as an add-on therapy or in combination with pridopidine by periodically administering the pharmaceutical composition and the pridopidine to the subject.

This invention also provides a pharmaceutical composition comprising an amount of pridopidine for use treating a subject afflicted with a neurodegenerative disorder as an add-on therapy or in combination with laquinimod by periodically administering the pharmaceutical composition and the laquinimod to the subject.

### DETAILED DESCRIPTION OF THE INVENTION

This invention provides a method of treating a human patient afflicted with a neurodegenerative disorder comprising periodically administering to the patient an amount of laquinimod and an amount of pridopidine, wherein the amounts when taken together are effective to treat the human patient.

In an embodiment of the present invention, the amount of laquinimod and the amount of pridopidine when taken together is more effective to treat the human patient than when each agent is administered alone. In another embodiment, each of the amount of laquinimod when taken alone, and the amount of pridopidine when taken alone is effective to treat the human patient. In another embodiment, either the amount of laquinimod when taken alone, the amount of pridopidine when taken alone, or each such amount when taken alone is not effective to treat the human patient.

In one embodiment, the neurodegenerative disorder is a trinucleotide repeat disorder. In another embodiment, the neurodegenerative disorder is a polyglutamine disease. In another embodiment, the neurodegenerative disorder is a proteinopathy. In another embodiment, the neurodegenerative disorder is Parkinson's disease, Alzheimer's disease, Amyotorphic lateral sclerosis (ALS) or Huntington's disease. In yet another embodiment, the neurodegenerative disorder is Huntington's disease.

In one embodiment, the amount of laquinimod and the amount of pridopidine when taken together is effective to reduce a symptom of the neurodegenerative disorder in the human patient. In another embodiment, the symptom is depression, anxiety, motor function impairment, cognitive impairment, a physical symptom, a mental symptom, an emotional symptom, a behavioral symptom, impairment of the patient's functional capacity or reduced lifespan. In another embodiment, the symptom is motor function impairment. In another embodiment, the motor function impairment is abnormal movements, myoclonic jerks, irregular movements of extremities, gait, facial grimacing, ataxia, inability to sustain motor act, hand movement or balance. In another embodiment, the patient's motor function is assessed by UHDRS, TMS or the modified motor score (mMS) derived from the Unified Huntington's Disease Rating Scale Total Motor Score (UHDRS, TMS). In yet another embodiment, the patient had an mMS score of 10 or greater at baseline.

In an embodiment of the present invention, the administration of laquinimod and pridopidine improves a symptom of the neurodegenerative disorder by at least 20%. In another embodiment, the administration of laquinimod and pridopidine improves a symptom of the neurodegenerative disorder by at least 30%. In another embodiment, the administration of laquinimod and pridopidine improves a symptom of the neurodegenerative disorder by at least 50%. In another embodiment, the administration of laquinimod and pridopidine improves a symptom of the neurodegenerative disorder by more than 100%. In another embodiment, the administration of laquinimod and pridopidine improves a symptom of the neurodegenerative disorder by more than 300%. In another embodiment, the administration of laquinimod and pridopidine improves a symptom of the neurodegenerative disorder by more than 1000%.

In one embodiment, the human patient is receiving laquinimod therapy prior to initiating pridopidine therapy. In another embodiment, the administration of laquinimod substantially precedes the administration of pridopidine. In another embodiment, the human patient is receiving pridopidine therapy prior to initiating laquinimod therapy. In another embodiment, the administration of pridopidine substantially precedes the administration of laquinimod.

In one embodiment, the administration of laquinimod is 0 minutes to 48 hours after the administration of pridopidine. In another embodiment, the administration of laquinimod is 3-5 hours after the administration of pridopidine. In another embodiment, the administration of pridopidine is 0 minutes to 48 hours after the administration of laquinimod. In another embodiment, the administration of pridopidine is 3-5 hours after the administration of laquinimod.

In one embodiment, laquinimod is laquinimod sodium. In another embodiment, the laquinimod is administered via oral administration. In another embodiment, the laquinimod is administered daily. In another embodiment, the laquinimod is administered more often than once daily. In another embodiment, the laquinimod is administered less often than once daily.

In one embodiment, the amount laquinimod administered is less than 0.6 mg/day. In another embodiment, the amount laquinimod administered is 0.1-40.0 mg/day. In another embodiment, the amount laquinimod administered is 0.1-2.5 mg/day. In another embodiment, the amount laquinimod administered is 0.25-2.0 mg/day. In another embodiment, the amount laquinimod administered is 0.5-1.2 mg/day. In another embodiment, the amount laquinimod administered is 0.25 mg/day. In another embodiment, the amount laquinimod administered is 0.3 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In another embodiment, the amount laquinimod administered is 1.2 mg/day. In another embodiment, the amount laquinimod administered is 1.5 mg/day. In another embodiment, the amount laquinimod administered is 2.0 mg/day.

In one embodiment, pridopidine is administered orally. In another embodiment, pridopidine is administered through an nasal, inhalation, subcutaneous, intravenous, intraperitoneal, intramuscular, intranasal, buccal, vaginal, rectal, intraocular, intrathecal, topical or intradermal route. In another embodiment, the pridopidine is administered daily. In another embodiment, the pridopidine is administered more often than once daily. In another embodiment, the administration of pridopidine is effected twice a day. In another embodiment, the pridopidine is administered less often than once daily.

In one embodiment, the amount pridopidine administered is 0.1-1000 mg/day. In another embodiment, the amount of pridopidine administered is greater than 135 mg/day. In another embodiment, the amount of pridopidine administered is 180-225 mg/day. In another embodiment, the amount pridopidine administered is 20-180 mg/day. In another embodiment, the amount pridopidine administered is 50-180 mg/day. In another embodiment, the amount pridopidine administered is 30-120 mg/day. In another embodiment, the amount pridopidine administered is 0.1-70 mg/day. In another embodiment, the amount pridopidine administered is 10-80 mg/day. In another embodiment, the amount pridopidine administered is 45-90 mg/day. In another embodiment, the amount pridopidine administered is 45 mg/day. In another embodiment, the amount pridopidine administered is 90 mg/day. In another embodiment, the amount pridopidine administered is about 45 mg/day. In another embodiment, the amount pridopidine administered is about 90 mg/day. In another embodiment, the amount pridopidine administered is less than 90 mg/day. In another embodiment, the amount pridopidine administered is less than 45 mg/day.

In one embodiment, a loading dose of an amount different form the intended dose is administered for a period of time at the start of the periodic administration. In another embodiment, the loading dose is double the amount of the intended dose. In another embodiment, the loading dose is half the amount of the intended dose.

In one embodiment, the method further comprises administration of an antidepressant, a psychotropic drug, an antipsychotic, amisulpride, haloperidol, olanzapine, risperidone, sulpiride, or tiapride. In an embodiment, the periodic administration of laquinimod and pridopidine continues for at least 3 days. In another embodiment, the periodic administration of laquinimod and pridopidine continues for more than 30 days. In another embodiment, the periodic administration of laquinimod and pridopidine continues for more than 42 days. In another embodiment, the periodic administration of laquinimod and pridopidine continues for 8 weeks or more. In another embodiment, the periodic administration of laquinimod and pridopidine continues for at least 12 weeks. In another embodiment, the periodic administration of laquinimod and pridopidine continues for at least 24 weeks. In another embodiment, the periodic administration of laquinimod and pridopidine continues for more than 24 weeks. In yet another embodiment, the periodic administration of laquinimod and pridopidine continues for 6 months or more.

This invention also provides a package comprising (a) a first pharmaceutical composition comprising an amount of laquinimod and a pharmaceutically acceptable carrier; (b) a second pharmaceutical composition comprising an amount of pridopidine and a pharmaceutically acceptable carrier; and (c) instructions for use of the first and second pharmaceutical compositions together to treat a human patient afflicted with a neurodegenerative disease. In an embodiment, the neurodegenerative disorder is Huntington's disease.

In one embodiment, the first pharmaceutical composition, the second pharmaceutical composition, or both the first and the second pharmaceutical composition are in the form of an aerosol or inhalable powder. In another embodiment, the first pharmaceutical composition, the second pharmaceutical composition, or both the first and the second pharmaceutical composition are in liquid form. In another embodiment, the first pharmaceutical composition, the second pharmaceutical composition, or both the first and the second pharmaceutical composition are in solid form. In another embodiment, the first pharmaceutical composition, the second pharmaceutical composition, or both the first and the second pharmaceutical composition are in capsule form. In another embodiment, the first pharmaceutical composition, the second pharmaceutical composition, or both the first and the second pharmaceutical composition are in tablet form. In another embodiment, the tablets are coated with a coating which inhibits oxygen from contacting the core. In another embodiment, the coating comprises a cellulosic polymer, a detackifier, a gloss enhancer, or pigment.

In one embodiment, the first pharmaceutical composition further comprises mannitol. In another embodiment, the first pharmaceutical composition further comprises an alkalinizing agent. In another embodiment, the alkalinizing agent is meglumine.

In one embodiment, the first pharmaceutical composition further comprises an oxidation reducing agent. In another embodiment, the first pharmaceutical composition is stable and free of an alkalinizing agent or an oxidation reducing agent. In another embodiment, the first pharmaceutical composition is free of an alkalinizing agent and free of an oxidation reducing agent. In another embodiment, the first pharmaceutical composition is stable and free of disintegrant.

In one embodiment, the first pharmaceutical composition further comprises a lubricant. In another embodiment, the lubricant is present in the composition as solid particles. In another embodiment, the lubricant is sodium stearyl fumarate or magnesium stearate.

In one embodiment, the first pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In yet another embodiment, the filler is mannitol or lactose monohydrate.

In an embodiment, the package further comprises a desiccant. In another embodiment, the desiccant is silica gel.

In one embodiment, the first pharmaceutical composition is stable has a moisture content of no more than 4%. In another embodiment, laquinimod is present in the composition as solid particles. In another embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In yet another embodiment, the oxygen absorbing agent is iron.

In an embodiment of the present invention, the amount of laquinimod in the first composition is less than 0.6 mg. In another embodiment, the amount of laquinimod in the composition is 0.1-40.0 mg. In another embodiment, the amount of laquinimod in the first composition is 0.1-2.5 mg. In another embodiment, the amount of laquinimod in the first composition is 0.25-2.0 mg. In another embodiment, the amount of laquinimod in the first composition is 0.5-1.2 mg. In another embodiment, the amount of laquinimod in the first composition is 0.25 mg. In another embodiment, the amount of laquinimod in the first composition is 0.3 mg. In another embodiment, the amount of laquinimod in the first composition is 0.5 mg. In another embodiment, the amount of laquinimod in the first composition is 0.6 mg. In another embodiment, the amount of laquinimod in the first composition is 1.0 mg. In another embodiment, the amount of laquinimod in the first composition is 1.2 mg. In another embodiment, the amount of laquinimod in the first composition is 1.5 mg. In another embodiment, the amount of laquinimod in the first composition is 2.0 mg.

In an embodiment of the present invention, the amount of pridopidine in the second composition is 0.1-1000 mg. In another embodiment, the amount of pridopidine in the second composition is 10-600 mg. In another embodiment, the amount of pridopidine in the second composition is 0.1-70 mg. In another embodiment, the amount of pridopidine in the second composition is 10-80 mg. In another embodiment, the amount of pridopidine in the second composition is 20-180 mg. In another embodiment, the amount of pridopidine in the second composition is 30-120 mg. In another embodiment, the amount of pridopidine in the second composition is 45-90 mg. In another embodiment, the amount of pridopidine in the second composition is 45 mg. In another embodiment, the amount of pridopidine in the second composition is 90 mg. In another embodiment, the amount of pridopidine in the second composition is about 45 mg. In another embodiment, the amount of pridopidine in the second composition is about 90 mg. In another embodiment, the amount of pridopidine in the second composition is less than 90 mg. In another embodiment, the amount of pridopidine in the second composition is less than 45 mg. In yet another embodiment, the amount of pridopidine in the second composition is 1, 5, 15, 20, 30, 50, 100, or 300 mg.

This invention also provides laquinimod for use as an add-on therapy or in combination with pridopidine in treating a human patient afflicted with a neurodegenerative disorder.

This invention also provides a pharmaceutical composition comprising an amount of laquinimod and an amount of pridopidine for use in treating a human patient afflicted with a neurodegenerative disorder, wherein the laquinimod and the pridopidine are to be administered simultaneously or contemporaneously. In an embodiment, the neurodegenerative disorder is Huntington's disease.

This invention also provides a pharmaceutical composition comprising an amount of laquinimod and an amount of pridopidine. In one embodiment, the pharmaceutical composition is in the form of an aerosol or inhalable powder. In an embodiment, the pharmaceutical composition is in liquid form. In an embodiment, the pharmaceutical composition is in solid form. In an embodiment, the pharmaceutical composition is in capsule form. In an embodiment, the pharmaceutical composition is in tablet form.

In one embodiment, the tablets are coated with a coating which inhibits oxygen from contacting the core. In another embodiment, the coating comprises a cellulosic polymer, a detackifier, a gloss enhancer, or pigment.

In one embodiment, the pharmaceutical composition further comprises mannitol. In another embodiment, the pharmaceutical composition further comprises an alkalinizing agent. In another embodiment, the alkalinizing agent is meglumine. In an embodiment, the pharmaceutical composition comprises an oxidation reducing agent.

In an embodiment the pharmaceutical composition is free of an alkalinizing agent or an oxidation reducing agent. In another embodiment, the pharmaceutical composition is free of an alkalinizing agent and free of an oxidation reducing agent.

In one embody, the pharmaceutical composition is stable and free of disintegrant. In another embodiment, the pharmaceutical composition further comprises a lubricant. In another embodiment, the lubricant is present in the composition as solid particles. In another embodiment, the lubricant is sodium stearyl fumarate or magnesium stearate.

In an embodiment, the pharmaceutical composition further comprises a filler. In another embodiment, the filler is present in the composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the amount of laquitiimod in the composition is less than 0.6 mg. In another embodiment, the amount of laquinimod in the composition is 0.1-40.0 mg. In another embodiment, the amount of laquinimod in the composition is 0.1-2.5 mg. In another embodiment, the amount of laquinimod in the composition is 0.25-2.0 mg. In another embodiment, the amount of laquinimod in the composition is 0.5-1.2 mg. In another embodiment, the amount of laquinimod in the composition is 0.25 mg. In another embodiment, the amount of laquinimod in the composition is 0.3 mg. In another embodiment, the amount of laquinimod in the composition is 0.5 mg. In another embodiment, the amount of laquinimod in the composition is 0.6 mg. In another embodiment, the amount of laquinimod in the composition is 1.0 mg. In another embodiment, the amount of laquinimod in the composition is 1.2 mg. In another embodiment, the amount of laquinimod in the composition is 1.5 mg. In another embodiment, the amount of laquinimod in the composition is 2.0 mg.

In an embodiment of the present invention, the amount of pridopidine is 0.1-1000 mg. In another embodiment, the amount of pridopidine is 10-600 mg. In another embodiment, the amount of pridopidine is 0.1-70 mg. In another embodiment, the amount of pridopidine is 10-80 mg. In another embodiment, the amount of pridopidine is 20-180 mg. In another embodiment, the amount of pridopidine is 30-120 mg. In another embodiment, the amount of pridopidine is 45-90 mg. In another embodiment, the amount of pridopidine is 45 mg. In another embodiment, the amount of pridopidine is 90 mg. In another embodiment, the amount of pridopidine is about 45 mg. In another embodiment, the amount of pridopidine is about 90 mg. In another embodiment, the amount of pridopidine is less than 90 mg. In another embodiment, the amount of pridopidine is less than 45 mg. In yet another embodiment, the amount of pridopidine is 1, 5, 15, 20, 30, 50, 100, or 300 mg.

This invention also provides use of an amount of laquinimod and an amount of pridopidine in the preparation of a combination for treating a human patient afflicted with a neurodegenerative disorder wherein the laquinimod or pharmaceutically acceptable salt thereof and the pridopidine are administered simultaneously or contemporaneously.

This invention also provides a pharmaceutical composition comprising an amount of laquinimod for use in treating a subject afflicted with a neurodegenerative disorder as an add-on therapy or in combination with pridopidine by periodically administering the pharmaceutical composition and the pridopidine to the subject.

This invention also provides a pharmaceutical composition comprising an amount of pridopidine for use treating a subject afflicted with a neurodegenerative disorder as an add-on therapy or in combination with laquinimod by periodically administering the pharmaceutical composition and the laquinimod to the subject.

In the methods, pharmaceutical compositions, packages, and uses as described herein, the laquinimod can be partly or fully deuterium-enriched. In an embodiment, laquinimod has deuterium enrichment of no less than about 10%. In another embodiment, laquinimod has deuterium enrichment of no less than about 50%. In another embodiment, laquinimod has deuterium enrichment of no less than about 90%. In another embodiment, laquinimod has deuterium enrichment of no less than about 98%. Deuterium-enriched forms of laquinimod are described in e.g., U.S. Pat. No. 8,252,933 and U.S. Patent Application Publication No. 2010/0055072, which are hereby incorporated by reference in their entireties into this application.

In the methods, pharmaceutical compositions, packages, and uses described herein, the pridopidine can be partly or fully deuterium-enriched. In an embodiment, pridopidine has deuterium enrichment of no less than about 10%. In another embodiment, pridopidine has deuterium enrichment of no less than about 50%. In another embodiment, pridopidine has deuterium enrichment of no less than about 90%. In another embodiment, pridopidine has deuterium enrichment of no less than about 98%. Deuterium-enriched forms of pridopidine are described in e.g., PCT International Application Publication Nos. WO 2012/028635 and WO 2011/107583, which are hereby incorporated by reference in their entireties into this application.

This invention also provides a therapeutic package for dispensing to, or for use in dispensing to, a subject afflicted with a neurodegenerative disorder or presenting a clinically isolated syndrome, which comprises: a) one or more unit doses, each such unit dose comprising: i) an amount of laquinimod and ii) an amount of pridopidine, wherein the respective amounts of said laquinimod and said pridopidine in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

For the foregoing embodiments, each embodiment disclosed herein is contemplated as being applicable to each of the other disclosed embodiments. In addition, the elements recited in the packaging and pharmaceutical composition embodiments can be used in the method and use embodiments described herein.

### Pridopidine

Pridopidine mixtures, compositions, the process for the manufacture thereof, the use thereof for treatment of various conditions, and the corresponding dosages and regimens are described in, e.g., PCT International Application Publication Nos. WO 2001/46145, WO 2011/107583, WO 2006/040155, U.S. Patent Application Publication No. 2011/0206782, U.S. Patent Application Publication No. 2010/0197712, each of which is hereby incorporated by reference in its entireties into this application.

### Laquinimod

Laquinimod mixtures, compositions, and the process for the manufacture thereof are described in, e.g., U.S. Pat. No. 6,077,851, U.S. Pat. No. 7,884,208, U.S. Pat. No. 7,989,473, U.S. Pat. No. 8,178,127, U.S. Application Publication No. 2010-0055072, U.S. Application Publication No. 2012-0010238, and U.S. Application Publication No. 2012-0010239, each of which is hereby incorporated by reference in their entireties into this application.

Use of laquinimod for treatment of various conditions, and the corresponding dosages and regimens, are described in U.S. Pat. No. 6,077,851 (multiple sclerosis, insulin-dependent diabetes mellitus, systemic lupus erythematosus, rheumatoid arthritis, inflammatory bowel disease, psoriasis, inflammatory respiratory disorder, atherosclerosis, stroke, and Alzhemier's disease), U.S. Application Publication No. 2011-0027219 (Crohn's disease), U.S. Application Publication No. 2010-0322900 (Relapsing-remitting multiple sclerosis), U.S. Application Publication No. 2011-0034508 (brain-derived neurotrophic factor (BDNF)-related diseases), U.S. Application Publication No. 2011-0218179 (active lupus nephritis), U.S. Application Publication No. 2011-0218203 (rheumatoid arthritis), U.S. Application Publication No. 2011-0217295 (active lupus arthritis), and U.S. Application Publication No. 2012-0142730 (reducing fatigue, improving quality of life, and providing neuroprotection in MS patients), each of which is hereby incorporated by reference in their entireties into this application.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit can be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar. Capsule or tablets can be easily formulated and can be made easy to swallow or chew; other solid forms include granules, and bulk powders.

Tablets may contain suitable binders, lubricants, disintegrating agents (disintegrants), coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248. These references in their entireties are hereby incorporated by reference into this application.

General techniques and compositions for making dosage forms useful in the present invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds).; Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject afflicted with a neurodegenerative disorder, e.g., HD, using laquinimod as an add-on or in combination with pridopidine which provides a more efficacious treatment than each agent alone. The use of laquinimod for certain neurodegenerative disorder, e.g., Huntington's disease, amyotrophic lateral sclerosis (ALS), and Alzheimer's disease had been previously suggested in, e.g., U.S. Patent Application Publication No. 2011-0034508. However, the inventors have surprisingly found that the combination of laquinimod and pridopidine is particularly effective for the treatment of neurodegenerative disorders such as HD as compared to each agent alone.

### Terms

As used herein, and unless stated otherwise, each of the following terms shall have the definition set forth below.

As used herein, “laquinimod” means laquinimod acid or a pharmaceutically acceptable salt thereof.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, “about” in the context of a numerical value or range means±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to Flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “combination” means an assemblage of reagents for use in therapy either by simultaneous or contemporaneous administration. Simultaneous administration refers to administration of an admixture (whether a true mixture, a suspension, an emulsion or other physical combination) of laquinimod and pridopidine. In this case, the combination may be the admixture or separate containers of laquinimod and pridopidine that are combined just prior to administration. Contemporaneous administration refers to the separate administration of laquinimod and pridopidine at the same time, or at times sufficiently close together that a synergistic activity relative to the activity of either laquinimod or pridopidine alone is observed.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding laquinimod therapy to a patient already receiving pridopidine therapy.

As used herein, “effective” when referring to an amount of laquinimod and/or pridopidine refers to the quantity of laquinimod and/or pridopidine that is sufficient to yield a desired therapeutic response without undue adverse side effects (such as toxicity, irritation, or allergic response) commensurate with a reasonable benefit/risk ratio when used in the manner of this invention.

“Administering to the subject” or “administering to the (human) patient” means the giving of, dispensing of, or application of medicines, drugs, or remedies to a subject/patient to relieve, cure, or reduce the symptoms associated with a disease, disorder or condition, e.g., a pathological condition.

“Treating” as used herein encompasses, e.g., inducing inhibition, regression, or stasis of a disease or disorder, e.g., Huntington's disease, or lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

A “symptom” associated with a neurodegenerative disorder includes any clinical or laboratory manifestation associated with the neurodegenerative disorder and is not limited to what the subject can feel or observe. For example, a symptom of Huntington's disease includes, but is not limited to, a patient's mMS, motor function as measured by, e.g., the UHDRS-TMS, cognitive function, anxiety and depression. “Improvement of” or “improving” a symptom as used herein refers to a favorable change in the patient's symptom as compared to baseline or as compared to a control subject not receiving the treatment.

As used herein, “substantially proceeds administration” means that the administration of one agent precedes another agent; and the two agents are not administered simultaneously or contemporaneously.

As used herein, “a subject afflicted with a neurodegenerative disorder” means a subject who has been clinically diagnosed to have the neurodegenerative disorder.

As used herein, a subject at “baseline” is as subject prior to administration of laquinimod or pridopidine.

“Polyglutamine disease” as used herein encompasses any inherited disorders characterized by an expanded CAG triplet repeat which codes for a long glutamine repeat including but not limited to Huntington's disease, spinobulbar muscular atrophy (SBMA), and dentatorubral pallidoluysian atrophy. Chai et al. (1999) “Analysis of the Role of Heat Shock Protein (Hsp) Molecular Chaperones in Polyglutamine Disease,” Journal of Neuroscience 19(23):10338-10347, which is hereby incorporated by reference in its entirety into this application.

“Proteinopathy” as used herein encompasses any disease caused by a misfolding and/or aggregation of proteins.

An HD patient's motor function can be assessed by the Unified Huntington's Disease Rating Scale (UHDRS) Motor Score or “modified motor score (mMS)” derived from the UHDRS Total Motor Score. UHDRS is a research tool which has been developed by the Huntington Study Group (HSG) to provide a uniform assessment of the clinical features and course of HD. The modified motor score is a modified version of the UHDRS made up of 19 items out of the 31 items on the UHDRS motor score. The modified Motor Score is made up of negative motor features such as bradykinesia, rigidity, hand function, eye movements, and gait. The 12 items not included in the mMS but included in the UHDRS motor score include chorea and dystonia, which may differ in their progression from the 19 items on the mMS. The UHDRS is described in, e.g., Huntington Study Group (1996) “Unified Huntington's Disease Rating Scale: Reliability and Consistency” Movement Disorders 11(2):136-142, which is hereby incorporated by reference in its entirety into this application.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Animal Models of Huntington's Disease

Most animal models of HD fall into two broad categories, genetic and non-genetic. Historically, nongenetic models have dominated the field of HD research, and typically induce cell death either by excitotoxic mechanisms of by disruption of mitochondrial machinery. Quinolinic acid and kainic acid have been the two most commonly used excitotoxic agents in both rodent and primate models of HD (Ramaswamy, 2007). Emerging molecular technology has enabled the development of genetic murine and, more recently, rat models that attempt to capture the hereditary nature of HD. There are two main categories of genetic mouse models, transgenic and knock-in. Transgenic mice results from the random insertion of a portion of the human htt gene, containing the polyglutamine repeat, in the mouse genome, the expression of which can be driven by different promoters. Alternatively, “knocking in” a portion of the human htt gene in the mouse htt gene locus on chromosome 7 results in the creation of knock-in mice. Transgenic models include transgenic mice model R6/2, R6/1, N171-82Q, YAC, and transgenic rat. Knock-in models include HdhQ92 mouse, HdhQ111 mouse, CAG140 mouse and CAG150 mouse (Ramaswamy, 2007).

### Example 1.1

### Toxin Models of HD

A quinolinic acid (QA) rat model is periodically administered an amount of laquinimod and an amount pridopidine. The periodic administration of laquinimod and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the rat than when pridopidine alone or laquinimod alone is administered at the same repetitive dose.

A 3-Nitro-propionic acid (3-NP) rat model is periodically administered an amount of laquinimod and an amount pridopidine. The periodic administration of laquinimod and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the rat than when pridopidine alone or laquinimod alone is administered at the same repetitive dose.

### Example 1.2

### Transgenic Models of HD

A R6/2 mouse model is periodically administered an amount of laquinimod and an amount pridopidine. The periodic administration of laquinimod and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the mouse than when pridopidine alone or laquinimod alone is administered at the same repetitive dose.

### Example 1.3

### Knock-In Mouse Models of HD

A CAG150 mouse model is periodically administered an amount of laquinimod and an amount pridopidine. The periodic administration of laquinimod and pridopidine is more effective (provides at least an additive effect or more than an additive effect) in preventing or attenuating weight loss, slowing, inhibiting, or reversing progression of motor, cognitive or behavioral symptoms, improving performance on the rotarod test, gait test, clasping test, and open-field test, slowing, inhibiting, or reversing progression of neurodegeneration in the brain, and prolonging survival, in the mouse than when pridopidine alone or laquinimod alone is administered at the same repetitive dose.

### Example 2

### Add-On Therapy for Treating Huntington'S Disease

Periodic oral administration of laquinimod (0.6 mg/day or 1.2 mg/day) as an add-on therapy for a human patient afflicted with HD who is already receiving pridopidine (45 mg once daily or 45 mg twice a day) provides a clinically meaningful advantage and is more effective (provides at least an additive effect or more than an additive effect) in treating the patient than when pridopidine is administered alone (at the same dose).

Periodic administration of pridopidine (45 mg once daily or 45 mg twice a day) as an add-on therapy for a human patient afflicted with HD who is already receiving laquinimod (0.6 mg/day or 1.2 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 3

### Add-On Therapy for Treating Huntington's Disease

Periodic oral administration of laquinimod (0.6 mg/day or 1.2 mg/day) as an add-on therapy for a human patient afflicted with HD who is already receiving pridopidine (67.5 mg once daily or 67.5 mg twice a day) provides a clinically meaningful advantage and is more effective (provides at least an additive effect or more than an additive effect) in treating the patient than when pridopidine is administered alone (at the same dose).

Periodic administration of pridopidine (67.5 mg once daily or 67.5 mg twice a day) as an add-on therapy for a human patient afflicted with HD who is already receiving laquinimod (0.6 mg/day or 1.2 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 4

### Add-On Therapy for Treating Huntington's Disease

Periodic oral administration of laquinimod (0.6 mg/day or 1.2 mg/day) as an add-on therapy for a human patient afflicted with HD who is already receiving pridopidine (90 mg once daily or 90 mg twice a day) provides a clinically meaningful advantage and is more effective (provides at least an additive effect or more than an additive effect) in treating the patient than when pridopidine is administered alone (at the same dose).

Periodic administration of pridopidine (90 mg once daily or 90 mg twice a day) as an add-on therapy for a human patient afflicted with HD who is already receiving laquinimod (0.6 mg/day or 1.2 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 5

### Add-On Therapy for Treating Huntington's Disease

Periodic oral administration of laquinimod (0.6 mg/day or 1.2 mg/day) as an add-on therapy for a human patient afflicted with HD who is already receiving pridopidine (112.5 mg once daily or 112.5 mg twice a day) provides a clinically meaningful advantage and is more effective (provides at least an additive effect or more than an additive effect) in treating the patient than when pridopidine is administered alone (at the same dose).

Periodic administration of pridopidine (112.5 mg once daily or 112.5 mg twice a day) as an add-on therapy for a human patient afflicted with HD who is already receiving laquinimod (0.6 mg/day or 1.2 mg/day) provides a clinically meaningful advantage and is more effective (provides at least an additive effect or more than an additive effect) in treating the patient than when laquinimod is administered alone (at the same dose).

The add-on therapies also provides efficacy (provides at least an additive effect or more than an additive effect) in treating the patient without undue adverse side effects or affecting the safety of the treatment:
1. The add-on therapy is effective (provides at least an additive effect or more than an additive effect) in improving symptoms of depression, sedation and anxiety.
2. The add-on therapy is effective (provides at least an additive effect or more than an additive effect) in slowing, inhibiting or reversing the progression of motor function and cognitive impairment.
3. The add-on therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The add-on therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The add-on therapy is effective (provides at least an additive effect or more than an additive effect) in improving the patient's functional capacity.
7. The add-on therapy is effective (provides at least an additive effect or more than an additive effect) in reducing, preventing progression of, or reversing mental, emotional and behavioral symptoms of HD.
8. The add-on therapy is effective (provides at least an additive effect or more than an additive effect) in prolonging the patient's lifespan.
9. The add-on therapy does not produce any significant side effects such as sedation and depression.

### Example 6

### Combination Therapy for Treating Huntington's Disease

HD is a fatal neurodegenerative disease characterized by uncoordinated and uncontrollable movements, cognitive deterioration, and behavioral and/or psychological problems. The classic onset of HD symptoms typically occurs in middle age, but the disease also manifests in children and the elderly. Disease progression is characterized by a gradual decline in motor control, cognition, and mental stability and generally results in death within 15-25 years of clinical diagnosis.

HD is a genetic disease, transmitted via autosomal-dominant inheritance. The defective gene, found on chromosome 4, causes the production of a mutant protein, huntingtin (Htt), which aggregates in the central nervous system (CNS) and results in the pathogenesis of HD. The prevalence of HD is approximately 10 per 100,000 in the US and Europe. The only currently marketed product in the United States indicated for HD is tetrabenazine, which has no effect on non-choreic symptoms and disease progression, and is associated with serious side effects such as suicidality and depression. Significant unmet medical needs remain in the development of alternative treatments for HD.

Huntexil® (pridopidine/ACR16) is a drug candidate being developed for the symptomatic treatment of hand movement, balance and gait disturbances in HD. Previous trials in the United States, Europe and Canada demonstrate significant symptomatic relief for patients with HD including improved hand movements and improved gait and balance. These results were observed without any side effects such as sedation and depression seen with other therapies such as neuroleptics and tetrabenzine.

Disclosed herein is the use of laquinimod in addition to or in combination with pridopidine for the treatment of HD.

Periodic oral administration of laquinimod (0.6 mg/day or 1.2 mg/day) in combination with pridopidine (45 mg once daily or 45 mg twice a day) to a human patient afflicted with HD provides increased efficacy (provides at least an additive effect or more than an additive effect) in treating the patient than when pridopidine is administered alone or when laquinimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impahinent.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### Example 7

### Combination Therapy For Treating Huntington's Disease

Disclosed herein is the use of laquinimod in addition to or in combination with pridopidine for the treatment of HD.

Periodic oral administration of laquinimod (0.6 mg/day or 1.2 mg/day) in combination with pridopidine (67.5 mg once daily or 67.5 mg twice a day) to a human patient afflicted with HD provides increased efficacy (provides at least an additive effect or more than an additive effect) in treating the patient than when pridopidine is administered alone or when laquinimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impairment.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### Example 8

### Combination Therapy for Treating Huntington's Disease

Disclosed herein is the use of laquinimod in addition to or in combination with pridopidine for the treatment of HD.

Periodic oral administration of laquinimod (0.6 mg/day or 1.2 mg/day) in combination with pridopidine (90 mg once daily or 90 mg twice a day) to a human patient afflicted with HD provides increased efficacy (provides at least an additive effect or more than an additive effect) in treating the patient than when pridopidine is administered alone or when laquinimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impairment.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### Example 9

### Combination Therapy For Treating Huntington's Disease

Disclosed herein is the use of laquinimod in addition to or in combination with pridopidine for the treatment of HD.

Periodic oral administration of laquinimod (0.6 mg/day or 1.2 mg/day) in combination with pridopidine (112.5 mg once daily or 112.5 mg twice a day) to a human patient afflicted with HD provides increased efficacy (provides at least an additive effect or more than an additive effect) in treating the patient than when pridopidine is administered alone or when laquinimod is administered alone (at the same dose). The combination therapy also provides efficacy (provides at least an additive effect or more than an additive effect) in treating the patient without undue adverse side effects or affecting the safety of the treatment.

The combination therapy provides a clinically meaningful advantage and is more effective (provides at least an additive effect or more than an additive effect) in treating the patient than when laquinimod or pridopidine is administered alone (at the same dose) in the following manner:
1. The combination therapy is effective (provides at least an additive effect or more than an additive effect) in improving symptoms of depression, sedation and anxiety.
2. The combination therapy is effective (provides at least an additive effect or more than an additive effect) in slowing, inhibiting or reversing the progression of motor function and cognitive impairment.
3. The combination therapy is effective (provides at least an additive effect or more than an additive effect) in reducing the severity of motor symptoms including abnormal movements, myoclonic jerks, irregular movements of extremities, lilting gait, gait disturbances, facial grimacing, ataxia, and inability to sustain motor act.
4. The combination therapy is effective (provides at least an additive effect or more than an additive effect) in improving the patient's hand movements, gait and balance.
5. The add-on therapy is effective (provides at least an additive effect or more than an additive effect) in slowing or preventing deterioration of or improving the patient's motor function as assessed by the modified motor score (mMS) derived from the Unified Huntington's Disease Rating Scale Total Motor Score (UHDRS, TMS).
6. The combination therapy is effective (provides at least an additive effect or more than an additive effect) in improving the patient's functional capacity.
7. The combination therapy is effective (provides at least an additive effect or more than an additive effect) in reducing, preventing progression of, or reversing mental, emotional and behavioral symptoms of HD.
8. The combination therapy is effective (provides at least an additive effect or more than an additive effect) in prolonging the patient's lifespan.
9. The combination therapy does not produce any significant side effects such as sedation and depression.

### REFERENCES

1. “Huntexil®” The NeurosSearch website, retrieved Sep. 24, 2012, <http://neurosearch.com/Default.aspx?ID=8172>.
2. “Huntington's disease”0 The NeurosSearch website, retrieved Sep. 24, 2012, <http://neurosearch.com/Default.aspx?ID=8172>.
3. Brod et al. (2000) Annals of Neurology, 47:127-131.
4. Brück (2011) “Insight into the mechanism of laquinimod action.” J Neurol Sci. 2011 July 15;306(1-2):173-9.
5. ClinicalTrials.gov—Open-label Extension Study of Pridopidine (ACR16) in the Symptomatic Treatment of Huntington Disease (OPEN-HART), retrieved Sep. 26, 2012, <http://www.clinicaltrials.gov/ct2/show/NCT01306929?term=NCT01306929&rank=1>.
6. de Yebenes et al., (2011) “Pridopidine for the treatment of motor function in patients with Huntington's disease (MermaiHD): a phase 3, randomized, double-blind, placebo-controlled trial,” The Lancet Neurology, 10(2): 1049-1057.
7. FDA 2005. Draft Guidance for Industry—Systemic Lupus Erythematosus—Developing Drugs for Treatment (http://vvwvv.fda.gov/dovvnloads/Drugs/GuidanceComplianceRegulatoryInformation/Guidanc es/ucm072063.pdf).
8. Filippi et al., Pridopidine reduces the proportion of MS lesions evolving into black holes, Neurol., 2001, 57:731-733.
9. Gurevich et al. (2010) “Laquinimod suppress antigen presentation in relapsing-remitting multiple sclerosis: in-vitro high-throughput gene expression study” (J Neuroimmunol. 2010 Apr. 15;221(1-2):87-94. Epub 2010 Mar. 27.
10. Huntington Study Group (1996) “Unified Huntington's Disease Rating Scale: Reliability and Consistency” Movement Disorders 11(2):136-142.
11. Kleinschmidt-DeMasters et al. (2005) New England Journal of Medicine, 353:369-379.
12. Langer-Gould et al. (2005) New England Journal of Medicine, 353:369-379.
13. Miller & Bezprozvanny (2010) “Corticostriatal circuit dysfunction in Huntington's disease: intersection of glutamate, dopamine and calcium,” Future Neurol, 5(5): 735-756.
14. Neuhaus et al. (2003) “Immunomodulation in multiple sclerosis: from immunosuppression to neuroprotection”, Trends Pharmacol Sci. 24:131-138.
15. NeuroSearch-The HART Study, retrieved Sep. 26, 2012 <hftp://neurosearch.com/Default.aspx?ID=8490>.
16. PCT International Application Publication No. WO 1998/30227, published Jul. 16, 1998.
17. PCT International Application Publication No. WO 2000/05250, published Feb. 3, 2000.
18. PCT International Application Publication No. WO 2000/18794, published Apr. 6, 2000.
19. PCT International Application Publication No. WO 2001/46145, published Jun. 28, 2001.
20. PCT International Application Publication No. WO 2003/048735, published Jun. 12, 2003.
21. PCT International Application Publication No. WO 2004/103297, published Dec. 2, 2004.
22. PCT International Application Publication No. WO 2006/016036, published Nov. 2, 2006.
23. PCT International Application Publication No. WO 2006/029393, published Mar. 16, 2006.
24. PCT International Application Publication No. WO 2006/029411, published Mar. 16, 2006.
25. PCT International Application Publication No. WO 2006/040155, published Apr. 20, 2006.
26. PCT International Application Publication No. WO 2006/083608, published Aug. 10, 2006.
27. PCT International Application Publication No. WO 2006/089164, published Aug. 24, 2006.
28. PCT International Application Publication No. WO 2006/116602, published Nov. 2, 2006.
29. PCT International Application Publication No. WO 2007/047863, published Apr. 26, 2007.
30. PCT International Application Publication No. WO 2007/047863, published Apr. 26, 2007.
31. PCT International Application Publication No. WO 2007/146248, published Dec. 21, 2007.
32. PCT International Application Publication No. WO 2009/070298, published Jun. 4, 2009.
33. PCT International Application Publication No. WO 2011/008274, published Jan. 20, 2011.
34. PCT International Application Publication No. WO 2011/022063, published Feb. 24, 2011.
35. PCT International Application Publication No. WO 2011/107583, published Sep. 9, 2011.
36. PCT International Application Publication No. WO 2012/051106, published Apr. 19, 2012.
37. Polman et al., (2005) “Diagnostic criteria for multiple sclerosis: 2005 revisions to the McDonald Criteria”, Annals of Neurology, Volume 58 Issue 6, Pages 840-846.
38. Polman et al., (2005) “Treatment with laquinimod reduces development of active MRI lesions in relapsing MS”, Neurology. 64:987-991.
39. Porter, Robert S. (2011). Huntington's disease. In The Merck manual of diagnosis and therapy (19^{th} ed.) pps 1763-1765.
40. Ramaswamy et al. (2007) “Animal Models of Huntington's Disease” ILAR Journal, 48(4):356-373.
41. RTT News Article dated April 12, 11, entitled “Teva Pharma, Active Biotech Post Positive Laquinimod Phase 3 ALLEGRO Results”.
42. Rudick et al. (2006) New England Journal of Medicine, 354:911-923.
43. Rudick, R. (1999) “Disease-Modifying Drugs for Relapsing-Remitting Multiple Sclerosis and Future Directions for Multiple Sclerosis Therapeutics”, Neurotherpatueics. 56:1079-1084.
44. Runström et al. (2006) “Inhibition of the development of chronic experimental autoimmune encephalomyelitis by laquinimod (ABR-215062) in IFN-β k.o. and wild type mice” Journal of Neuroimmunologv, 173(2006):69-78.
45. Sandberg-Wollheim et al. (2005) “48-week open safety study with high-dose oral laquinimod in patients”, Mult Scler. 11:S154 (Abstract).
46. Tyagi et al., (2010) “Symptomatic Treatment and Management of Huntington's Disease: An Overview” Global Journal of Pharmacology, 4(1):06-12.
47. U.S. Patent Application Publication 2010-0197712, published Aug. 5, 2010 (Carlsson et al.).
48. U.S. Patent Application Publication No. 2008-0207526, published Aug. 28, 2008 (Strominger et al.).
49. U.S. Patent Application Publication No. 2010-0322900, published Dec. 23, 2010 (Tarcic et al.).
50. U.S. Patent Application Publication No. 2011-0027219, published Feb. 3, 2011 (Tarcic et al.).
51. U.S. Patent Application Publication No. 2011-0034508, published Feb. 10, 2011 (Liat Hayardeny).
52. U.S. Patent Application Publication No. 2011-0206782, published Aug. 25, 11 (Zhang).
53. U.S. Patent Application Publication No. 2011-0217295, published Sep. 8, 2011 (Haviv and Tarcic).
54. U.S. Patent Application Publication No. 2011-0218179, published Sep. 8, 2011 (Haviv and Tarcic).
55. U.S. Patent Application Publication No. 2011-0218203, published Sep. 8, 2011 (Joel Kaye et al.).
56. U.S. Patent Application Publication No. 2012-0010238, published Jan. 12, 2012 (Fristedt).
57. U.S. Patent Application Publication No. 2012-0010239, published Jan. 12, 2012 (Piryatinsky et al.).
58. U.S. Patent Application Publication No. 2012-0142730, published Jun. 7, 2012 (Tarcic et al.).
59. U.S. Pat. No. 3,849,550, issued Nov. 19, 1974 (Teitelbaum et al).
60. U.S. Pat. No. 5,800,808, issued Sep. 1, 1998 (Konfino et al).
61. U.S. Pat. No. 5,858,964, issued Jan. 12, 1999 (Aharoni et al).
62. U.S. Pat. No. 5,981,589, issued Nov. 9, 1999 (Konfino et al).
63. U.S. Pat. No. 6,048,898, issued Apr. 11, 2000 (Konfino et al).
64. U.S. Pat. No. 6,054,430, issued Apr. 25, 2000 (Konfino et al).
65. U.S. Pat. No. 6,077,851, issued Jun. 20, 2000 (Bjork et al).
66. U.S. Pat. No. 6,214,791, issued Apr. 10, 2001 (Arnon et al).
67. U.S. Pat. No. 6,342,476, issued Jan. 29, 2002 (Konfino et al).
68. U.S. Pat. No. 6,362,161, issued Mar. 26, 2002 (Konfino et al).
69. U.S. Pat. No. 7,566,767, issued Jul. 28, 2009 (Strominger et al.).
70. U.S. Pat. No. 7,589,208, issued Sep. 15, 2009 (Jansson et al).
71. U.S. Pat. no. 7,884,208, issued Feb. 8, 2011 (Frenkel et al.).
72. U.S. Pat. No. 7,989,473, issued Aug. 2, 2011 (Patashnik et al.).
73. U.S. Pat. No. 8,178,127, issued May 15, 2012 (Safadi et al.).
74. U.S. Pat. No. 8,252,993, issued Aug. 28, 2012 (Gant and Shahbaz).
75. Venuto et al., (2012) “Pharmacologic Approaches to the Treatment of Huntington's Disease,” Movement Disorders 27(1): 31
76. Vollmer et al. (2008) “Pridopidine after induction therapy with mitoxantrone in relapsing multiple sclerosis” Multiple Sclerosis, 00:1-8.
77. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.
78. Yong (2002) “Differential mechanisms of action of interferon-β and pridopidine in MS” Neurology, 59:1-7.
79. Yong (2002) “Differential mechanisms of action of interferon-β and pridopidine in MS” Neurology, 59:1-7.

1. A method of treating a human patient afflicted with a neurodegenerative disorder comprising periodically administering to the patient an amount of laquinimod and an amount of pridopidine, wherein the amounts when taken together are effective to treat the human patient.

2. The method of claim 1, wherein the amount of laquinimod and the amount of pridopidine when taken together is more effective to treat the human patient than when each agent is administered alone.

3. The method of claim 1, wherein each of the amount of laquinimod when taken alone, and the amount of pridopidine when taken alone, is effective to treat the human patient.

4. The method of claim 1, wherein either the amount of laquinimod when taken alone, or the amount of pridopidine when taken alone is not effective to treat the human patient.

5. The method of claim 1, wherein the neurodegenerative disorder is a polyglutamine disease.

6. The method of claim 1, wherein the neurodegenerative disorder is a proteinopathy.

7. The method of claim 1, wherein the neurodegenerative disorder is Parkinson's disease, Alzheimer's disease, Amyotorphic latreal sclerosis (ALS) or Huntington's diseasse.

8. The method of claim 7, wherein the neurodegenerative disorder is Huntington's disease.

9. The method of claim 1, wherein the amount of laquinimod and the amount of pridopidine when taken together is effective to reduce a symptom of the neurodegenerative disorder in the human patient.

10-14. (canceled)

15. The method of claim 1, wherein the administration of laquinimod and pridopidine improves a symptom of the neurodegenerative disorder by at least 20%-1000%.

16-20. (canceled)

21. The method of claim 1, wherein the human patient is receiving laquinimod therapy prior to initiating pridopidine therapy.

22. The method of claim 21, wherein the administration of laquinimod precedes the administration of pridopidine.

23. The method of claim 1, wherein the human patient is receiving pridopidine therapy prior to initiating laquinimod therapy.

24. The method of claim .23, wherein the administration of pridopidine precedes the administration of laquinimod.

25-30. (canceled)

31. The method of claim 1, wherein the amount laquinimod administered is 0.1-40.0 mg/day.

32. The method of claim 31, wherein the amount laquinimod administered is 0.1-2.5 mg/day.

33-48. (canceled)

49. The method of claim 1, wherein the amount pridopidine administered is 20-180 mg/day.

50-55. (canceled)

56. The method of claim 1, wherein a loading dose of an amount different from the intended dose is administered for a period of time at the start of the periodic administration.

57-67. (canceled)

68. A package comprising
(a) a first pharmaceutical composition comprising an amount of laquinimod and a pharmaceutically acceptable carrier;
(b) a second pharmaceutical composition comprising an amount of pridopidine and a pharmaceutically acceptable carrier; and
(c) instructions for use of the first and second pharmaceutical compositions together to treat a human patient afflicted with a neurodegenerative disease.

69-124. (canceled)

125. A pharmaceutical composition comprising an amount'of laquinimod and an amount of pridopidine.

126-176. (canceled)
